(19) United States
(12) Patent Application Publication
(10) Pub. No.: US 2026/0096823 A1
(43) Pub. Date: Apr. 9, 2026

(54) THREE-DIMENSIONAL PRINTED DISPOSABLE PATELLA CLAMP

(71) Applicants: Smith & Nephew, Inc., Memphis, TN (US); Smith & Nephew Orthopaedics AG, Zug (CH)

(72) Inventors: Thomas Joseph Francis, Cordova, TN (US); Wil Thornton, Cordova, TN (US); Ruxandra Cristiana Marinescu Tanasoca, Memphis, TN (US)

(73) Assignees: Smith & Nephew, Inc., Memphis, TN (US); Smith & Nephew Orthopaedics AG, Zug (CH)

(21) Appl. No.: 19/347,114

(22) Filed: Oct. 1, 2025

Related U.S. Application Data
(60) Provisional application No. 63/703,376, filed on Oct. 4, 2024.

(51) Int. Cl. A61B 17/15; A61B 17/16; A61B 17/17; B33Y 80/00

(52) U.S. Cl. CPC A61B 17/158; A61B 17/1677; A61B 17/1767; B33Y 80/00

(57) ABSTRACT

Disclosed herein is a disposable patella clamp. In some examples, the patella clamp is configured as a patient specific patella clamp that is custom designed to fit the anatomy of a particular patient. A patient undergoes diagnostic imaging which provides patient specific data including patella diameter and thickness, patella shape, and limb alignment. The imaging can be used to generate a 3D digital model of the patient's anatomy associated with the patellar region. The patella clamp can be designed and manufactured based on the model as an integrated clamp using an additive manufacturing process with biocompatible materials. The patella clamp may include one or more tracking elements operative to enable the patella clamp to be used with a computer-assisted and/or robotic surgical system.

### CROSS-REFERENCE TO RELATED APPLICATIONS

This application is a non-provisional of, and claims the benefit of the filing date of, U.S. Provisional Patent Application Ser. No. 63/703,376, filed Oct. 4, 2024, and titled “3D Printed Disposable Patella Clamp,” the entire contents of which application is hereby incorporated by reference in its entirety.

### TECHNICAL FIELD

The present disclosure is directed to an orthopedic surgical instrument, and, more specifically to a patient specific, disposable patella clamp.

### BACKGROUND

Anterior knee pain after Total Knee Arthroplasty (“TKA”) is one of the most frequent patellofemoral complications and the incidence of reported post-op pain is exceedingly high. This is one of the most severe and frustrating complications in orthopedics, as problems with the patella tracking still account for half of the poor outcomes after TKA. There are many factors that may affect patellar tracking in TKA, including implant factors, implant position, and orientation. Errors in surgical technique are the most common cause of patellar maltracking, asymmetrical patellar cut, incorrect patellar resection depth, incorrect sizing, malposition of the patellar component, etc. Currently, there is a large variation in the preferred method for patella preparation, and the optimal management of the patella during TKA remains controversial. Some surgeons prefer conventional instruments for patellar resection, while many surgeons prefer a freehand technique, in which anatomic landmarks are used to estimate the patellar resection depth/thickness and symmetry.

Surgeons may elect to perform patella resection using either a free hand technique or using conventional instrumentation. In a free hand patella resection, a patient's knee is flexed, and the patella is everted. The patella is held in place with surgical clamps or forceps. The surgeon measures the patella thickness with calipers and makes the patella resection free hand with a saw. The surgeon then uses a drill guide to determine the size of the patella and drills lug holes.

Alternatively, a surgeon may elect patella resection using conventional instrumentation, in which several metal instruments are used to resect the patella. A metal instruments patella tray is provided, that must be sterilized and prepared for the surgery whether or not all of the instruments are used. The patella tray usually contains a large, heavy metal patella clamp, patellar depth stops, reamers, different sizes collets, drill guides, a caliper, and several patellar trials.

Orthopedic patella clamps are known in the art. In general, a patella clamp includes a handle assembly and a clamping, gripping, or resection assembly (terms used interchangeably herein without the intent to limit) and are typically large and heavy. One disadvantage of known patella clamps is that surgeons must manually adjust the handle assembly and/or the clamping assembly to correspond to a patient's specific anatomy during surgery. As such, using the known patella clamps for patella preparation is cumbersome and time consuming.

It would be beneficial to provide a patient specific patella clamp. A patient specific patella clamp would improve a surgeon's accuracy and reduce surgical time related to adjusting the patella clamp to match a specific patient's anatomy. A patient specific patella clamp would remove the need for calipers/measurements during surgery as well as the preparation of a patella instrumentation tray.

In addition, and/or alternatively, it would be beneficial to provide disposable, single use patella clamps. A disposable, single use patella clamp would provide advantages over conventional metal patella clamps in terms of saving money, time and space by reducing lead times, limited or no inventory, reducing sterilization costs, reducing room turn-over time, reducing surgical time and the risk of infection, and overall reducing the total procedure cost.

Accordingly, there remains a need for patient specific disposable patella clamp.

### BRIEF SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein is a patient specific 3D printed disposable patella clamp. In use, the patella clamp is configured to grip and secure a patient's patella. The patella clamp is customized to fit the unique anatomy of a particular patient. As a disposable clamp, the patella clamp requires less space in the operating room compared to a tray of conventional instruments, reduces the cost of sterilization, reduces the time to clean and manage instrument sets, and reduces the chance of infection to patients. A patient undergoes diagnostic imaging to collect patient specific data used to generate a 3D digital model of the patellar region. The patella clamp can be customized to match the dimensions and/or features of the 3D digital model.

Disclosed herein is a surgical system configured to generate a virtual model of a patient's patella. The surgical system may include or may use a patella clamp with a tracking frame. The surgical system may include a tracking system communicatively coupled to at least one tracking frame coupled to the patella clamp. In use, the patella clamp is arranged and configured to grip the patient's patella. The surgical system may execute a computer-assisted surgical application operative to generate patella information based on tracking information received from the at least one tracking frame. The patella information may be associated with at least one patellar characteristic, such as location, dimensions, cutting planes, and/or the like. The patella model may be presented via a display device and configured to be a virtual representation of the patient's patella. In any preceding or subsequent example, the at least one tracking frame may include a tracking array of optical sensors for use with an optical tracking system.

A disposable patella clamp may include a handle assembly with a first handle affixed to a second handle by a hinging mechanism; an engagement plate disposed on a distal end of the second handle; a guide assembly disposed on a distal end of the first handle; a guidepost disposed on a surface of one of the first or second handles and configured to interact with an opening formed in the other one of the first and second handles; and wherein when a force is applied to the handle assembly, a patient's patella is secured between the guide assembly and the engagement plate with the guidepost passing through the opening formed in the handle.

In any preceding or subsequent example, the patella clamp may be an integral additive manufactured patella clamp.

In any preceding or subsequent example, the patella clamp may be manufactured using one or more of nylon, biocompatible photopolymer resin (BioMed Clear), thermoplastics, and engineered polymers.

In any preceding or subsequent example, the patella clamp may be a patient specific patella clamp.

In any preceding or subsequent example, the hinging mechanism may be a living hinge.

In any preceding or subsequent example, the patella clamp may include a second guidepost extending from a surface on a proximal end of one of the first and second handles, the second guidepost configured to interact with a second opening formed in the other one of the first and second handles.

In any preceding or subsequent example, the hinging mechanism may include one or more springs.

In any preceding or subsequent example, the one or more springs may include one or more of a coil spring, helical spring, barrel spring, hourglass spring, conical spring, magazine spring, volute spring, flat spring, and torsion spring.

In any preceding or subsequent example, the guide assembly may be a patient specific guide assembly corresponding to patient imaging.

In any preceding or subsequent example, the guide assembly may include a drill guide with one or more drill guide openings where one or more of a diameter of the drill guide, a layout of the one or more drill guide openings, and a diameter of the one or more drill guide openings are based on the patient imaging.

In any preceding or subsequent example, the guide assembly may include a cutting guide, where one or more of a height and a depth of the cutting guide are based on patient imaging.

In any preceding or subsequent example, a central axis of the guide assembly may align with a longitudinal axis of the handle assembly.

In any preceding or subsequent example, a central axis of the guide assembly may be disposed at an angle relative to a longitudinal axis of the handle assembly.

In any preceding or subsequent example, the central axis of the guide assembly may be disposed at a 90-degree angle clockwise or counterclockwise relative to the longitudinal axis of the handle assembly.

In any preceding or subsequent example, the engagement plate may include one or more of teeth, grooves, indentations, and ridges on a surface interior to the patella clamp configured to restrict movement of the patient's patella when secured between the guide assembly and the engagement plate.

In any preceding or subsequent example, the guidepost may include a threaded knob, a plate, or a cam configured to interact with the opening formed in the other one of the first and second handles to secure a position of the patella clamp.

In any preceding or subsequent example, the guide assembly is configured to be interchangeable and affixable to the handle assembly with one or more fastening mechanisms.

In any preceding or subsequent example, the engagement plate is configured to be interchangeable and affixable to the handle assembly with one or more fastening mechanisms.

In any preceding or subsequent example, a method for securing a patient's patella within a patella clamp includes providing a patella clamp, the patella clamp including: a handle assembly with a first handle affixed to a second handle by a hinging mechanism, an engagement plate disposed on a distal end of the first handle; a guide assembly disposed on a distal end of the second handle; and a guidepost extending from a surface of one of the first and second handles, the guidepost configured to interact with an opening formed in the other one of the first and second handles; inserting a patella between the guide assembly and the engagement plate; and applying a force to the handle assembly, the force causing the guidepost to pass through the opening to secure a position of the patella clamp.

In any preceding or subsequent example, a computer-assisted surgical system, includes at least one computing device, which includes: a display device; processing circuitry; and a memory coupled to the processing circuitry, the memory may include instructions that, when executed by the processing circuitry, cause the processing circuitry to: receive tracking information associated with a patella detected by at least one tracking element coupled to a tracking patella clamp engaging the patella, determine patella information based on the tracking information, the patella information indicating at least one characteristic of the patella, generate a patella model configured as a virtual representation of the patella configured based on the patella information, and present a graphical user interface (GUI) object visually depicting the patella model on a GUI screen via the display device.

Preceding or subsequent examples of the present disclosure provide numerous advantages. In one non-limiting example advantage, the patella clamp may be a patient specific patella clamp that is custom designed to fit the unique anatomy and needs of a particular patient. The patient specific patella clamp is an intuitive, user-friendly patella clamp that is disposable, low cost, and light weight allowing a surgeon to make accurate patellar cuts. As a disposable patient specific clamp, the patella clamp requires less space in the operating room compared to a tray of conventional instruments, reduces the cost of sterilization, reduces the time to clean and manage instrument sets, and reduces the chance of infection to patients. The patient specific patella clamp removes the need for calipers for measurements during surgery as the patella clamp drill guide and cutting guide are customized to a patient's anatomy based on the patient's imaging and 3D digital model.

Preceding or subsequent examples of the present disclosure provide numerous technological advantages and technical features over conventional systems. One non-limiting example of a technological advantage may include providing a patella clamp with a tracking frame of the present disclosure that may be used in connection with a computer-assisted and/or robotic surgical system. Another non-limiting example of a technological advantage may include providing a surgical system configured to allow a surgeon to visualize a patella preparation step and/or performance information associated with a patella preparation step prior to making any adjustments to the actual patient patella. Surgical planning processes according to any preceding or subsequent example may, among other things, facilitate planning of patella preparation and/or implant device positioning with decreased complexity and improved efficiency and effectiveness as compared to conventional systems. For example, surgical planning processes according to any preceding or subsequent example may allow healthcare professionals to avoid invalid patella preparation steps and/or avoid erroneous or ineffective patella preparations during the planning and/or performance of a surgical procedure, saving time and effort and improving patient outcomes. Preceding or subsequent examples are not limited in this context. Additional technological advantages and technical features over conventional systems would be known to those of skill in the art based on the present disclosure.

Further features and advantages of at least some of the preceding or subsequent examples of the present invention, as well as the structure and operation of various preceding or subsequent examples of the present invention, are described in detail below with reference to the accompanying drawings.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced. By way of illustration, various examples of the disclosed device will now be described, with reference to the accompanying drawings, in which:

FIG. 1 illustrates a perspective view of an example of a patella clamp in accordance with one of more features of the present disclosure.

FIG. 2A illustrates a perspective view of an alternate example of a patella clamp in accordance with one of more features of the present disclosure.

FIG. 2B illustrates an alternate perspective view of the patella clamp depicted in FIG. 2A in accordance with one of more features of the present disclosure.

FIG. 3A illustrates a perspective view of an alternate example of a patella clamp in accordance with one of more features of the present disclosure.

FIG. 3B illustrates an alternate perspective view of the patella clamp depicted in FIG. 3A in accordance with one of more features of the present disclosure.

FIG. 3C illustrates an alternate perspective view of the patella clamp depicted in FIGS. 3A-3B in accordance with one of more features of the present disclosure.

FIG. 4 illustrates a perspective view of an alternate example of a patella clamp in accordance with one of more features of the present disclosure.

FIG. 5 illustrates a perspective view of an alternate example of a patella clamp in accordance with one of more features of the present disclosure.

FIG. 6 illustrates a perspective view of an alternate example of a patella clamp with modular engagement elements in accordance with one of more features of the present disclosure.

FIG. 7 illustrates an example of a tracking frame in accordance with one of more features of the present disclosure.

FIG. 8 illustrates a system for performing a surgical procedure using a computer-assisted and/or robotic system in accordance with one of more features of the present disclosure.

FIG. 9 is a block diagram depicting an operating environment for planning and/or performing a surgical procedure in accordance with one of more features of the present disclosure.

### DETAILED DESCRIPTION

Various features of a 3D printed disposable patella clamp will now be described more fully hereinafter with reference to the accompanying drawings, in which one or more features of the patella clamp will be shown and described. It should be appreciated that the various features may be used independently of, or in combination, with each other. It will be appreciated that a patella clamp as disclosed herein may be embodied in many different forms and should not be construed as being limited to the examples set forth herein. Rather, these examples are provided so that this disclosure will convey certain features of the patella clamp to those skilled in the art.

The patella clamp may be a patient specific patella clamp that is custom designed to fit the unique anatomy and needs of a particular patient. The patient specific patella clamp is an intuitive, user-friendly patella clamp that is disposable, low cost, and light weight allowing a surgeon to make accurate patellar cuts. As a disposable patient specific clamp, the patella clamp requires less space in the operating room compared to a tray of conventional instruments, reduces the cost of sterilization, reduces the time to clean and manage instrument sets, and reduces the chance of infection to patients.

In use, the patella clamp is configured to grip and secure a patient's patella. The patella clamp is designed provide the strength to clamp and/or hold the patella. As a patient specific patella clamp, the patella clamp is customizable to a patient's patella sizes, i.e., diameter and/or thickness, and the desired patellar resection depth.

In some examples, the patient undergoes diagnostic imaging such CT, MRI, X-ray, ultrasound, or any other suitable imaging technique. The imaging provides patient specific data including patella diameter and thickness, patella shape, and limb alignment. The imaging can be used to generate a 3D digital model of the patient's anatomy associated with the patellar region.

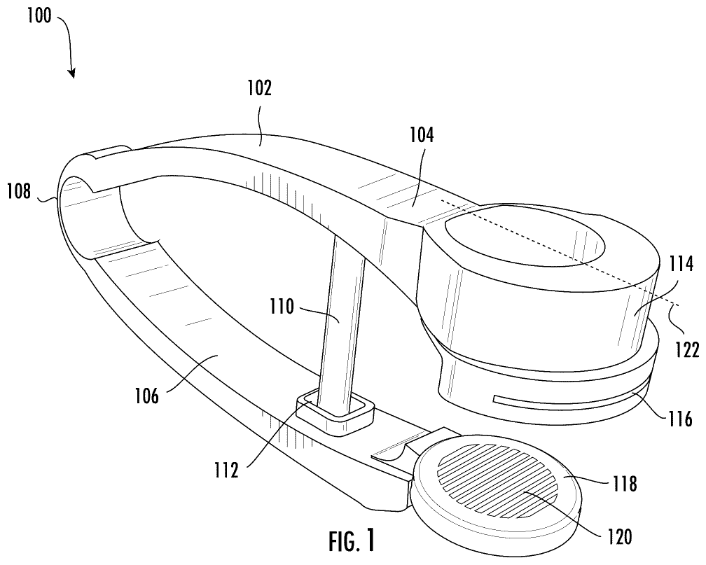

Based on the 3D model, dimensions and/or features of the various elements of the patella clamp, described in greater detail herein with reference to FIGS. 1-6, can be customized to conform with the patient's anatomy associated with the patellar region. In some examples, the patella clamp can be customized to a patella diameter and a patella thickness of a particular patient. In some examples, the gripping end, including the guide assembly, cutting guide, engagement plate, and drill guide, of the patella clamp is customized to conform with the patient's diagnostic imaging. As the patella clamp is custom manufactured for a patient, any features and/or dimensions of the patella clamp can be customized. For example, a length, width, and/or thickness of the handle assembly; a size and/or rigidity, i.e., flexibility, of the hinging mechanism, a diameter of the guide assembly, an angle of orientation of the guide assembly, a height and/or depth of the cutting guide, an angle of orientation of the cutting guide, i.e., an angle at which a saw would be inserted into the cutting guide, a size and shape of the engagement plate, the type and orientation of the texture on the textured surface; a diameter and/or thickness of the drill guide, a quantity, orientation/layout, and diameter of the drill guide openings; the size and/or shape of the patella engaging members; and any other dimensions and/or features of the various elements of the patella clamp. The patient specific patella clamp removes the need for calipers for measurements during surgery as the patella clamp drill guide and cutting guide are customized to a patient's anatomy based on the patient's imaging and 3D digital model.

The patient specific patella clamp can be manufactured as an integrated, i.e., single component, clamp using an additive manufacturing process, such as 3D printing. The patella clamp can be manufactured using various biocompatible materials, such as nylon, biocompatible photopolymer resin (BioMed Clear), thermoplastics, engineered polymers, any other suitable biocompatible 3D printing materials, and any combinations thereof. In some examples, the preferred material is nylon. In some examples, the biocompatible materials are sterilizable. The patella clamp may be manufactured using an additive manufacturing process capable of varying the mechanical properties of the various elements of the patella clamp. For example, mechanical properties can be varied across the elements of the patella clamp by controlling printing parameters such as infill density and pattern, layer height, and print orientation; and materials such as multi-material printing and dual-curable resins. Controlling the mechanical properties of the patella clamp can provide differing rigidity, stiffness, and/or flexibility in the elements of the patella clamp. For example, the hinging mechanism can be made more flexible as compared to the guide assembly, cutting guide, drill guide, and engagement plate, which may need to be more rigid as necessitated by the functionality of the element.

In addition, and/or alternatively, in accordance with one or more features of the present disclosure, the patella clamp may include one or more tracking elements operative to enable the patella clamp to be used with a computer-assisted and/or robotic surgical system to, among other things, enable modeling, visualization, and/or the like of the patella and/or resulting preparation steps to the patella.

Referring to FIGS. 1-6, examples of a patella clamp 100 in accordance with one or more features of the present disclosure will now be shown and described.

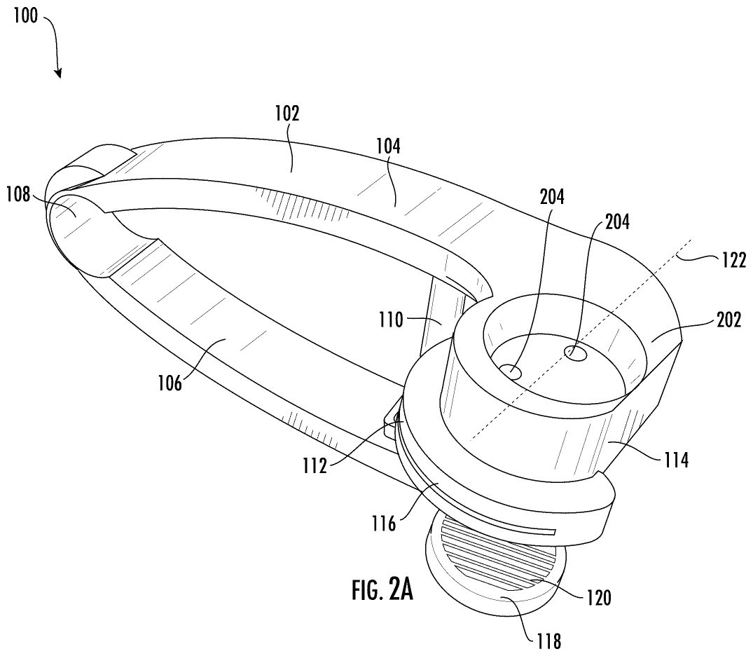

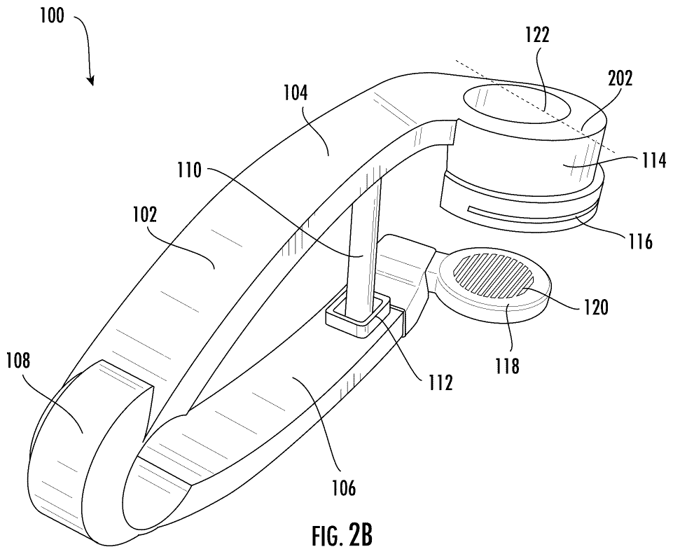

FIG. 1 illustrates a perspective view of an example of a patella clamp 100, in accordance with one of more features of the present disclosure. As shown, the patella clamp 100 includes a handle assembly 102 with an upper or first handle 104 connected to a lower or second handle 106 by a hinging mechanism 108. In the example of FIG. 1, the hinging mechanism is a living hinge, an integral hinge, or any other suitable flexible hinging connection. A guidepost 110 extends from a lower surface of the upper handle 104 and engages, passes through, etc. with a lower handle opening 112, although this is but one configuration and the guidepost 110 may extend from an upper surface of the lower handle 106 and engages, passes through, etc. with an opening formed in the upper handle 104. In some examples, the guidepost 110 may be located in a center region of the upper handle 104. In an alternate example, the guidepost 110 may be located nearer a distal end of the upper handle 104. The patella clamp 100 includes a gripping end comprised of guide assembly 114 with cutting guide 116 and engagement plate 118, although this is but one configuration and the engagement plate may have other shapes and configurations. As previously described herein, the features and/or dimensions of the gripping end may be patient specific aligned to the patient's diagnostic imaging. In an example, the guide assembly 114 may have a diameter of 32 mm or any other suitable size to conform to a specific patient's patella. The guide assembly 114 may also include a drill guide 202 as depicted and described with reference to FIG. 2A. In some examples, the guide assembly 114 is a bone contacting surface to hold the patella, provide a cutting slot for a saw blade, and provide a drill guide for lug holes. In some examples, the cutting guide 116 has a customized internal depth stop and a customized external saw slot to accommodate a desired resection height allowing a surgeon to make a smooth, symmetrical, planar cut. In an example, the cutting guide 116 may be a straight opening or an angled opening configured to accommodate a surgical saw.

In some examples and as depicted in FIG. 1, the guide assembly 114 may be disposed at the distal end of the upper handle 104 relative to the hinging mechanism 108 such that a central axis 122 of the guide assembly 114 aligns with a longitudinal axis of the handle assembly 102. The patella clamp 100 has an engagement plate 118. The engagement plate 118 is disposed at the distal end of the lower handle 106 relative to the hinging mechanism 108. In some examples, engagement plate 118 has a textured surface 120 facing the interior of the patella clamp 100. The textured surface 120 is configured to engage with a patient's patella to restrict movement of the patella when the patella clamp 100 is secured. In some examples, the textured surface 120 of the engagement plate 118 includes teeth, grooves, indentations, ridges, any other suitable surface texture, and any combination thereof. In an alternate example and not depicted in FIG. 1, a patella clamp may be a similar device as compared patella clamp 100 for non-PSI (patient specific instrumentation) procedures which may include features to allow adjustment of a cutting guide height and a dial to adjust an angle of the cutting guide.

In some examples and for each of the examples described herein, a patient's patella is placed within the opening between the guide assembly 114 and the engagement plate 118. When in use, a surgeon applies a force to the handle assembly 102 of the patella clamp 100. The force compresses the hinging mechanism 108 causing a distance between the guide assembly 114 and the engagement plate 118 to decrease until the patella is secured within the patella clamp 100. In some examples, the guidepost 110 extends through the lower opening 112. In an alternate example, the patella is secured within the patella clamp 100 by engagement of the guidepost 110 with the lower handle opening 112. In some examples, the guidepost 110 has a threaded knob, a plate, a cam, or similar engagement element configured to secure, lock, etc. the position of the patella clamp 100. With the patella secured within patella clamp 100, a surgeon may proceed to cut the patella through the cutting guide 116 and drill lug holes through the drill guide openings 204. In some examples, the surgeon may cut the patella prior to drilling the lug holes. In an alternate example, the surgeon may drill the lug holes prior to cutting the patella.

FIG. 2A illustrates a perspective view of an alternate example of a patella clamp 100, in accordance with one of more features of the present disclosure. FIG. 2A depicts elements described herein with reference to FIG. 1. FIG. 2A depicts a patella clamp 100 with a guide assembly 114 with a drill guide 202 with one or more drill guide openings 204. In some examples, the drill guide 202 has three drill guide openings 204. Alternately, the drill guide 202 can include any quantity of drill guide openings 204 necessary for securing an implant to a patient's patella. In some examples and as depicted in FIG. 2A, the central axis 122 of the guide assembly 114 is disposed at an angle, i.e., curved, relative to the longitudinal axis of the handle assembly 102. The angle conforms to a surgical approach for ease of use. The guide assembly 114 may be disposed orthogonally relative to the longitudinal axis of the handle assembly 102 in either a clockwise or counterclockwise direction. In some examples, the angle of the guide assembly 114 may be based on a right knee versus a left knee TKA. In an alternate example, the guide assembly 114 may be disposed at any angle relative to the longitudinal axis of the handle assembly 102 depending on surgeon preference.

In some examples, each of the one or more drill guide openings 204 may be of a same size, i.e., a same diameter and depth. Alternatively, a diameter of the individual drill guide openings 204 may vary based on the patient's imaging results.

FIG. 2B illustrates an alternate perspective view of the patella clamp 100 depicted in FIG. 2A, in accordance with one of more features of the present disclosure. FIG. 2B depicts elements described herein with reference to FIG. 1 and FIG. 2A.

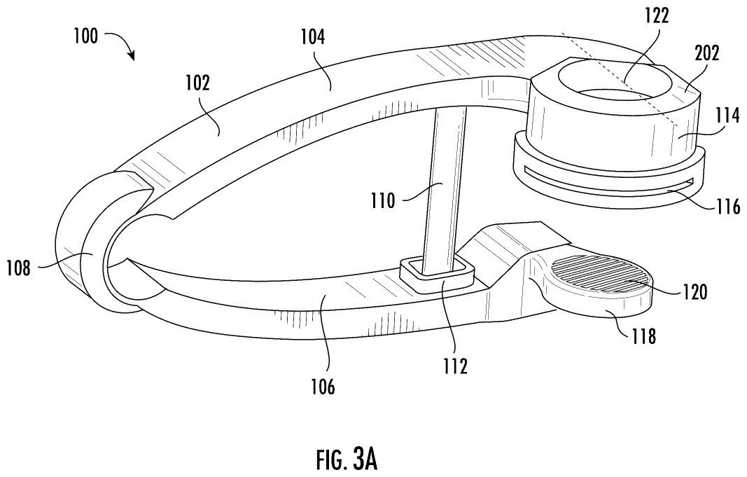

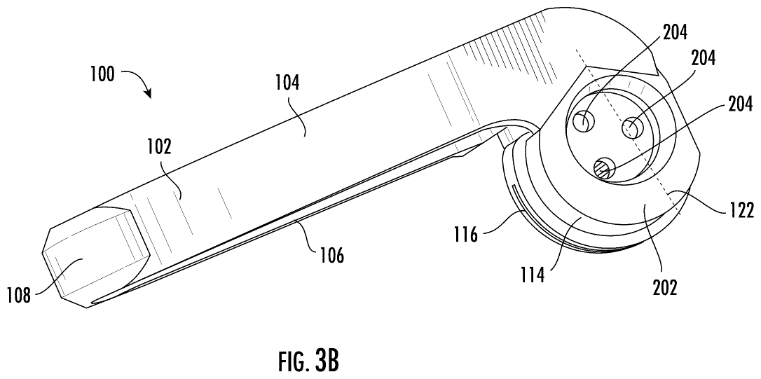

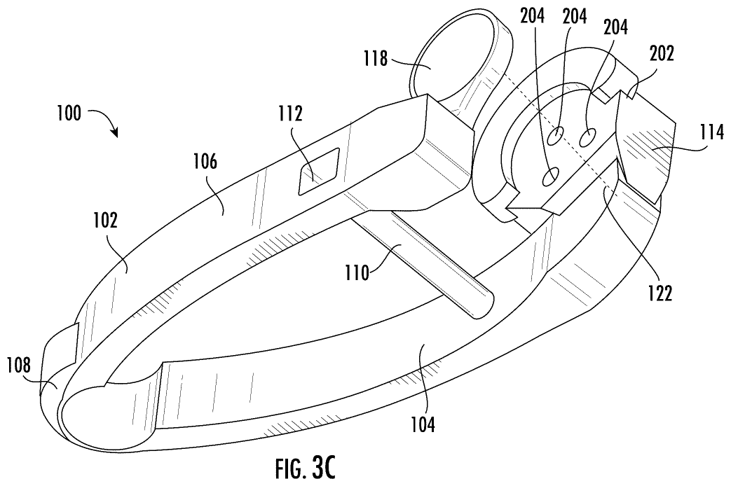

FIG. 3A illustrates a perspective view of an alternate example of a patella clamp 100 in accordance with one of more features of the present disclosure. FIG. 3A depicts elements described herein with reference to FIG. 1 and FIG. 2A. In an example, the guide assembly 114 may have a diameter of 35 mm or any other suitable size to conform to a specific patient's patella.

FIG. 3B illustrates an alternate perspective view of the patella clamp 100 depicted in FIG. 3A, in accordance with one of more features of the present disclosure. FIG. 3B depicts elements described herein with reference to FIG. 1 and FIG. 2A.

FIG. 3C illustrates an alternate perspective view of the patella clamp 100 depicted in FIGS. 3A-3B, in accordance with one of more features of the present disclosure. FIG. 3C depicts elements described herein with reference to FIG. 1 and FIG. 2A. FIG. 3C depicts a bottom view of the patella clamp 100 illustrating the lower handle opening 112.

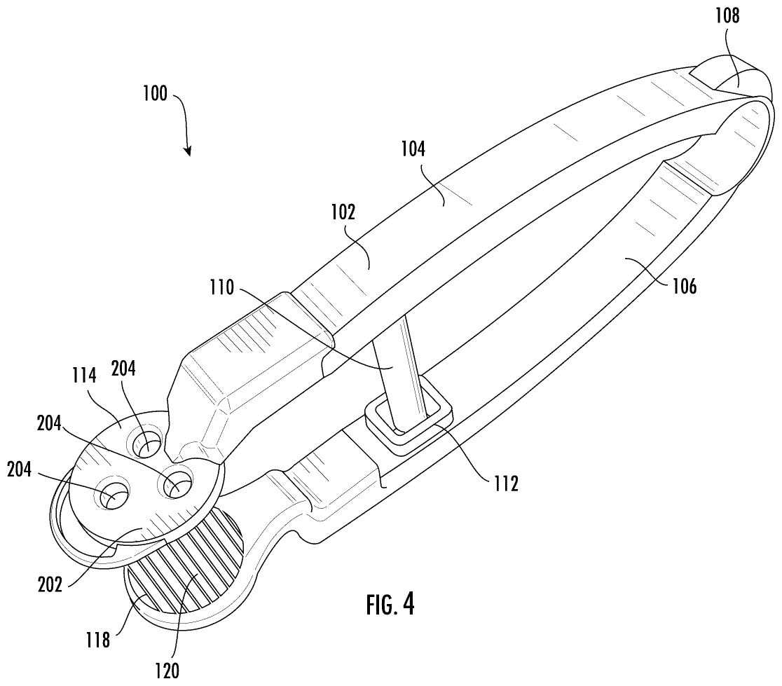

FIG. 4 illustrates a perspective view of an alternate example of a patella clamp 100, in accordance with one of more features of the present disclosure. FIG. 4 depicts elements described herein with reference to FIG. 1 and FIG. 2A. In the example depicted in FIG. 4, the patella clamp 100 comprises guide assembly 114 with a drill guide 202 with drill guide openings 204 absent a cutting guide 116. In an example, the patella clamp 100 as depicted in FIG. 4 may be used in combination with a PSI resection guide which does not contain drill holes. In an example, the patella clamp 100 as depicted in FIG. 4 may be used for conventional and/or robotic surgical procedures.

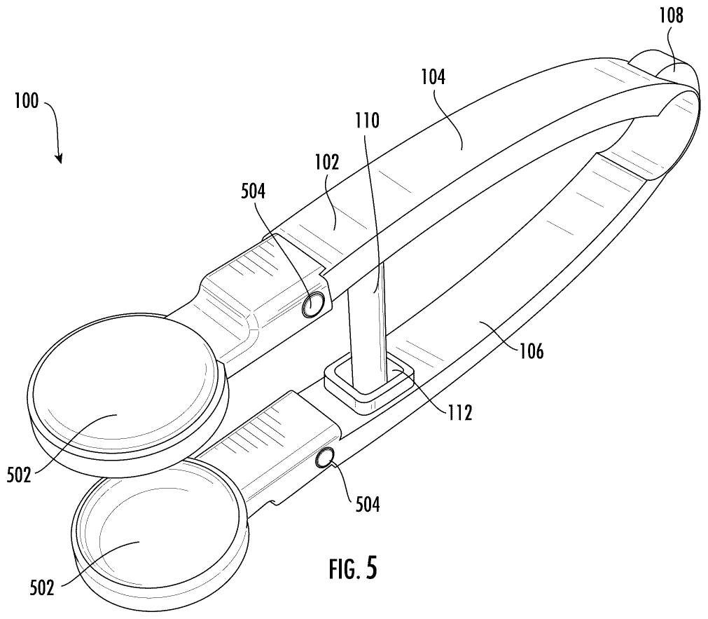

FIG. 5 illustrates a perspective view of an alternate example of a patella clamp 100 with modular engagement elements, in accordance with one of more features of the present disclosure. FIG. 5 depicts elements described herein with reference to FIG. 1 and FIG. 2A. FIG. 5 depicts an example of a patella clamp 100 where the engagement members on the distal end of the handle assembly 102 relative to the hinging mechanism 108 are modular, removable, and interchangeable. FIG. 5 depicts patella engaging members 502 affixed to the handle assembly 102 by fastening mechanisms 504. In an example, the patella clamp 100 as depicted in FIG. 5 may be used in combination with a PSI resection guide that does not contain surface contours for clamping a patella implant, either cemented or porous, onto the prepared surface, i.e., the bony surface. In an example, the patella clamp 100 as depicted in FIG. 5 may be used for conventional and/or robotic surgical procedures.

Fastening mechanisms 504 can be any type of fastener to secure the patella engaging members 502 to the handle assembly 102. In some examples, fastening mechanisms 504 may be snap fasteners, rivets, clips, pins, detent pins, or any other suitable fastening device. While two fastening mechanisms 504 are depicted, any suitable number of fastening mechanisms 504 may be used. For example, additional fastening mechanisms 504 may be used on the opposing side of the handle assembly 102. In an alternate example, the patella engaging members 502 may snap or otherwise be secured within an opening in the ends of the upper handle 104 and lower handle 106.

While FIG. 5 depicts patella engaging members 502 as modular engagement elements, any of the engagement elements previously described herein may be modular and affixed to the handle assembly 102 by fastening mechanisms 504 including guide assembly 114, drill guide 202, cutting guide 116, and engagement plate 118.

In some examples, handle assembly 102 may be reusable with engaging members 502 being modular patient specific engaging members. As previously described above, any of the engagement elements previously described herein may be modular and affixed to the reuseable handle assembly 102 including guide assembly 114, drill guide 202, cutting guide 116, and engagement plate 118.

In an alternate example, the patella clamp 100 depicted in FIG. 5 may be an integral clamp as described herein with reference to FIGS. 1-4, i.e., the patella engaging members are not modular but are integral to the patella clamp 100.

In some examples, the patella clamp 100 depicted in FIG. 5 is configured to clamp and seat an implant while surgical cement solidifies to affix the implant to the patella. The patella clamp 100 is used to secure a patient's patella to an implant with patella engaging members 502. A surgeon can apply a force, i.e., pressure, to the handle assembly 102 to fully seat the implant on the patella surface and secure the implant on the patella surface.

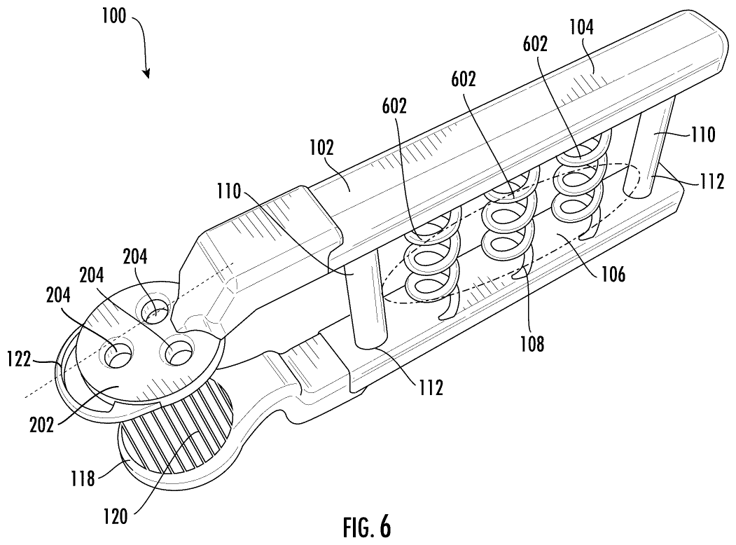

FIG. 6 illustrates a perspective view of an alternate example of a patella clamp 100, in accordance with one of more features of the present disclosure. FIG. 6 depicts elements described herein with reference to FIG. 1 and FIG. 2A. As shown, the patella clamp 100 includes a handle assembly 102 with an upper handle 104 connected to a lower handle 106 by a hinging mechanism 108. Guideposts 110 extend from a lower surface of the upper handle 104 and engage with lower handle openings 112. In the example of FIG. 6, the hinging mechanism 108 includes one or more springs 602. While three springs 602 are depicted, any suitable number of springs 602 could be used, for example, one or two springs 602. In some examples, springs 602 are one or more of a coil spring, helical spring, barrel spring, hourglass spring, conical spring, magazine spring, volute spring, flat spring, torsion spring, or any other suitable type of spring. In an alternate example, the hinging mechanism 108 may include a central spring designed as a leaf spring or a double leaf spring.

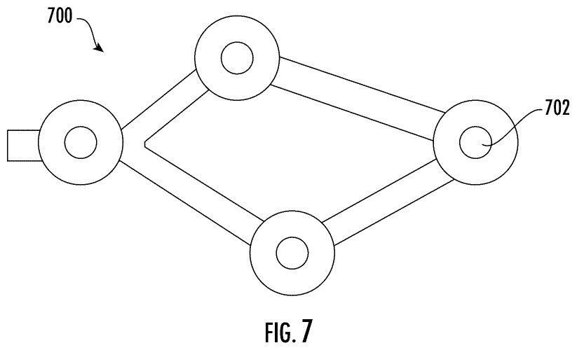

FIG. 7 illustrates a tracking frame 700, in accordance with one of more features of the present disclosure. In some examples, the patella clamp 100 may be in the form of a “tracking patella clamp” that includes one or more tracking frames 700, as depicted in FIG. 7. In any preceding or subsequent example, the tracking frame 700 may include a plurality or array of tracking elements 702 of devices, sensors, and/or the like operative to provide for tracking, navigation, determination of orientation, determination of position, and/or the like of a portion of the patient (for instance, a patella) in a computer-assisted and/or robotic surgical system. In any preceding or subsequent example, the tracking elements 702 may be or may include fiducial markers, markers, trackers, sensors, and/or the like (terms used interchangeably herein without the intent to limit). In any preceding or subsequent example, tracking frame 700 and/or tracking elements 702 may be, may include, and/or may be coupled to one or more transceivers (for instance, a wireless transmitting device) configured to transmit information detected by tracking frame 700 and/or tracking elements 702, for example, to a computing device.

In any preceding or subsequent example, tracking frame 700 and/or tracking elements 702 may be coupled to handle assembly 102 and, as a result, may be configured to rotate with handle assembly 102. In such a “rotating” example, a tracking element may be associated with a fixed assembly and/or patella to provide a reference (or “triangulate”) for tracking frame 700 and/or tracking elements 702 so that the tracking information may be collected and may be correct in any possible rotational orientation. In another “rotating” example, an offset (for instance, based on the position of handle assembly 102) may be determined in order to format the tracking information to correspond to the actual position of tracking frame 700 and/or tracking elements 702.

A tracking patella clamp configured according to any preceding or subsequent example may allow a surgeon the ability to use reamers, saw blades, and/or other preparation tools in combination with computer-assisted and/or robotic surgery techniques. For example, with a tracker attached to the resection jaw of a tracking patella clamp, the surgeon may be able to visualize the planar cut on a patella (and/or other patella preparation) and interaction with femoral (and/or other anatomy and/or components, such as an implant component) prior to making any cuts or other preparation steps.

In any preceding or subsequent example, tracking elements 702 may be coupled to various parts of patella clamp 100. Tracking elements 702 may be attached to any portion of patella clamp 100, including, without limitation, handle assembly 102, guide assembly 114, drill guide 202, cutting guide 116, or any other portions of the patella clamp 100.

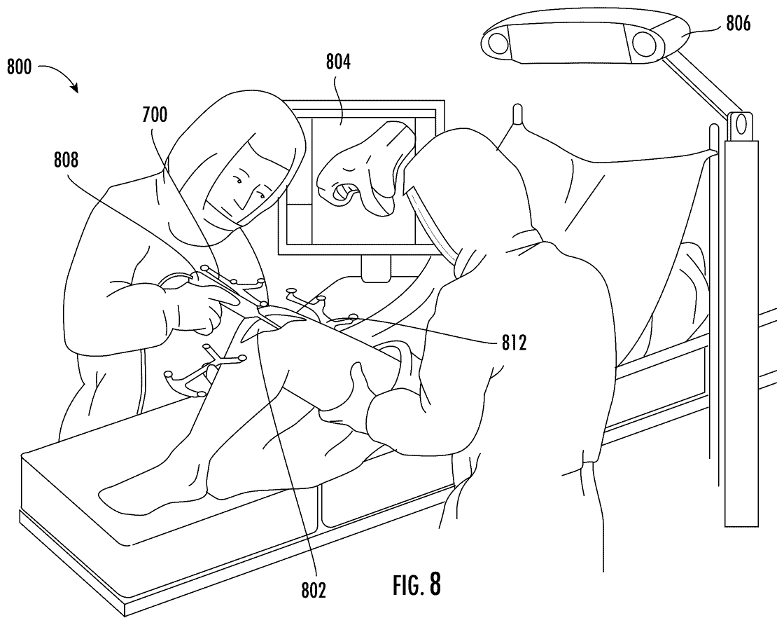

FIG. 8 illustrates a system 800 for performing a surgical procedure using a computer-assisted and/or robotic system, in accordance with one of more features of the present disclosure. In any preceding or subsequent example, system 800 may be or may include an image-free (for instance, CT-less) system. In other examples, system 800 may be or may include an image-based system based on diagnostic image data. In any preceding or subsequent example, system 800 may operate using a combination of image-free and image-based processes. Preceding or subsequent examples are not limited in this context.

System 800 may include a surgical cutting tool 808, or surgical tool, with an associated tracking frame 700 (also referred to as a tracking array 810), graphical user interface 804, a tracking system 806, and patient tracking frames 812 (for instance, one or more trackers, tracking arrays, and/or the like). The illustration also includes an incision 802 through which, for example, a knee revision surgery may be performed.

In some examples, the illustrated robotic surgical system 800 depicts a hand-held computer-controlled surgical robotic system, for instance, the same or similar to the Cori® and/or Navio® Surgical System from Smith & Nephew, Inc. of Cordova, Tennessee, United States. System 800 may use a tracking system 806, or other type of tracking system, coupled to a robotic controller to track and control a hand-held surgical instrument. For example, tracking system 806 tracks tracking array 810 coupled to surgical cutting tool 808 and tracking arrays 810 coupled to the patient to track locations of the instrument relative to the target bone (see, for example, FIG. 8).

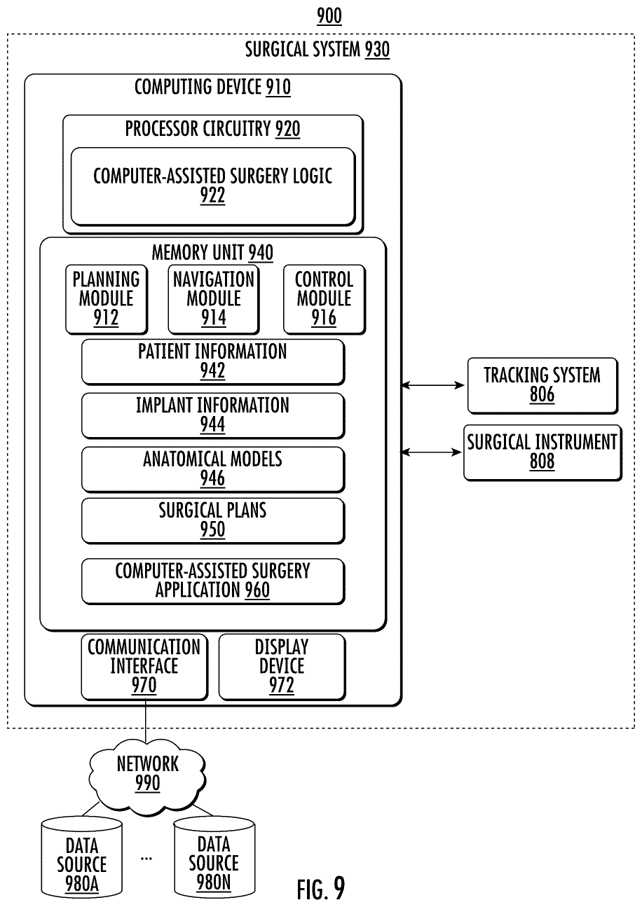

FIG. 9 is a block diagram depicting an operating environment 900 for planning and/or performing a surgical procedure according to an example. A non-limiting example of a surgical procedure may include an orthopedic or arthroplasty procedure.

As shown in FIG. 9, operating environment 900 may include a surgical system 930. In any preceding or subsequent example, surgical system 930 may include a computing device 910 communicatively coupled to network 990 via a communication interface (for instance, a transceiver) 970. Computing device 910 may be or may include one or more logic devices, including, without limitation, a server computer, a client computing device, a personal computer (PC), a workstation, a laptop, a notebook computer, a smart phone, a tablet computing device, and/or the like. Although a single computing device 910 is depicted in FIG. 9, preceding or subsequent examples are not so limited, as surgical system 930 may include and/or operably communicate with, and surgery planning processes according to any preceding or subsequent example may be performed using, a plurality of computing devices 910. In addition, components of computing device 910 depicted in FIG. 9 may be arranged within a plurality of different computing devices. In any preceding or subsequent example, surgical system 930 may be or may be a part of system 800 of FIG. 8. Preceding or subsequent examples are not limited in this context.

In any preceding or subsequent example, surgical system 930 may include computing device 910 operating as or operating as part of a control system (for instance, system 800), a tracking system 806, a surgical instrument, such as surgical cutting tool 808, and/or the like. Optionally, in any preceding or subsequent example, surgical system 930 may also include or may be operatively coupled to a display device 972 and/or a one or more data sources (for instance, databases) 980a-n. In any preceding or subsequent example, display device 972 and/or databases 980a-n may be used to provide navigation and control of a surgical instrument 808, which may include navigation and control of a cutting tool, a point probe, a bur, a bone saw, a reamer, or other tools/instruments, that may be used during an arthroplasty procedure, such as an orthopedic (or similar) prosthetic implant surgery.

Control system 910 may include one or more computing devices configured to coordinate information received from tracking system 806 and provide control to surgical instrument 808. In any preceding or subsequent example, control system 910 may include a planning module 912, a navigation module 914, a control module 916, and a communication interface 970. In any preceding or subsequent example, planning module 912 may provide pre-operative planning services that allow surgeons or other healthcare professionals to virtually plan a procedure prior to reshaping the patella (and other portions of the knee joint) during an implant procedure on the patient.

In some examples, such as for a total knee arthroplasty (“TKA”) procedure, planning module 912 may be used to generate and/or present a virtual model of a patella (“patella model”). The patella model may be manipulated in reference to a virtual implant host model (for instance, a “knee joint model”) and/or virtual implant device (“implant model”).

In any preceding or subsequent example, the virtual model of the implant host (for instance, the joint to be revised, including the patella, and/or components thereof) may be created through use of a point probe or similar instrument tracked by optical tracking system 806. Control system 910, for instance, via planning module 912, may be used to collect data (patella information) from surfaces of the patella to create a patella model in the form of a virtual model of the patient's actual anatomical structure. This method may, among other things, increase the accuracy of the planning process by using data collected from the patient's actual patella to allow for, among other things, the accurate and/or iterative reshaping of the patella to ensure proper fit of new prosthetic implants and optimization of anatomical alignment. The patella model may be generated based on the patella information, including, without limitation, width, height, maximum thickness, medial facet thickness, lateral facet thickness, bone quality, highest point, lowest point, and/or the like.

In some examples, navigation module 914 may coordinate tracking the location and orientation of the implant, the implant host (for instance, the patient patella, femur, tibia, and/or the like), and/or surgical instrument surgical cutting tool 808. In certain examples, navigation module 914 may also coordinate tracking of the virtual models (for instance, a patella model) used during pre-operative or intra-operative planning within planning module 912. Tracking the virtual models may include operations such as alignment of the virtual models with the implant host, generating predicted performance information, through data obtained via tracking system 806. In these examples, navigation module 914 receives input from tracking system 806 regarding the physical location and orientation of surgical instrument 808 and the patella of the patient. Tracking of the implant host may include tracking multiple individual bone structures, such as with tracking arrays, such as tracking frame 700. For example, during a TKA procedure, tracking system 806 may track the patella using tracking frame 700 engaged with the patella.

In some examples, control module 916 may process information provided by navigation module 914 to generate control signals for displaying patella information on display 972 and/or controlling surgical instrument 808. In certain examples, control module 916 may also work with navigation module 914 to produce visual animations to assist the surgeon during an operative procedure. Visual animations may be displayed via a display device, such as display device 972. In some examples, the visual animations may include real-time or substantially real-time 3D representations of the implant, the patella, the femur, the tibia, and/or surgical instrument 808, among other things.

In any preceding or subsequent example, markers (such as reflective markers), sensors, tracking elements (“patella tracking elements”), and/or the like may be arranged on or around the patella for use by tracking elements 702 to determine tracking information. The tracking information may be used by a computer-assisted surgery system (see, for example, FIG. 8) to determine patella information. The patella tracking elements may have known positions, locations, orientations, and/or the like.

In another example, the surgeon may be able to determine a characteristic (for instance, thickness) of the patella following a preparation step (for instance, burring a specified amount from a portion of the patella).

In an additional example, surgical system 930 may facilitate the use of burring instead of the use of a saw and saw guide. For example, surgical system 930 may continuously monitor how much bone is being taken off and/or how much is being retained. The surgeon may probe the high point of the patella and instruct surgical system 930 how much bone to keep or to take away. In one instance, after planar resection, the peg holes for the patella could be made by a burr and/or reamer.

In a further example, surgical system 930 may show the surgeon the resulting planar surface, for instance, via a collet attached to the resection guide. In this example, appropriate depth stops may be used to control depth. If a tracker were attached to the reamer or other preparation tool, depth control could be controlled via computer-assisted surgery application 960. For example, the difference between the marker on the resection guide and the reamer could tell the reamed depth.

In another example, if a surgeon specifies a biconvex patella implant, the biconvex surface and patella peg hole could be burred automatically via a computer-assisted and/or robotic surgical system.

Referring back to FIG. 9, in any preceding or subsequent example, communication interface 970 may facilitate communication between control system 910 and external systems and devices. Communication interface 970 may include both wired and wireless communication interfaces, such as Ethernet, IEEE 802.11 wireless, or Bluetooth, among others. As illustrated in FIG. 9, in this example, the primary external systems connected via the communication interface 970 may include a tracking system 806 (which may be or may include patella tracking functionality according to any preceding or subsequent example) and a surgical instrument 808. Although not shown, databases 980a-n and the display device 972, among other devices, may also be connected to control system 910 via communication interface 970. In some examples, communication interface 970 communicates over an internal bus to other modules and hardware systems within control system 910.

In any preceding or subsequent example, tracking system 806 may provide location and orientation information for surgical devices and parts of an implant host's anatomy, including a patella registered within surgical system 930 according to any preceding or subsequent example, to assist in navigation and control of semi-active robotic surgical devices. One illustrative and non-limiting example tracking system 806 may be the OPTOTRAK® 3-D motion and position measurement and tracking system described herein, although those of ordinary skill in the art will recognize that other tracking systems of other accuracies and/or resolutions may be used.

In some examples, a surgeon may use surgical instrument 808 to shape the patella to accommodate a prosthetic implant. For instance, control surgical system 930 may track and control a surgical cutting instrument to perform precise cuts, burring, reaming, or other preparations of the patella. In any preceding or subsequent example, a surgeon may enter a preparation instruction via GUI interface (such as cut or burr X mm from the top of the patella, ream distance Y mm into location Z on patella, and/or the like) and surgical system 930 may provide control signals to the corresponding robotic surgical tool to implement the preparation instruction on the patient patella.

In any preceding or subsequent example, control system 910 may operate to generate and/or access virtual or anatomical models of a patient. For example, control system 910 may operate to generate anatomical models of a patient via an anatomical model generation process the same or similar to the methods described in U.S. Patent Application Publication No. 2019/0365474, titled “Systems and Methods for Planning and Performing Image Free Implant Revision Surgery,” and/or PCT International Application No. PCT/US2020/054231, titled “Registration of Intramedullary Canal During Revision Total Knee Arthroplasty,” both of which are incorporated by reference in the present disclosure as if fully written herein. Preceding or subsequent examples are not limited in this context. For example, surgical planning processes according to any preceding or subsequent example may generate and/or access anatomical models created via various other processes (for instance, image-based processes) capable of operating with any preceding or subsequent example described in the present disclosure.

The anatomical models may be or may include virtual representations of portions of the patient, such as patella, for example, the same or similar to the computer models of patient anatomy provided in the Cori® and/or Navio® surgical systems. The anatomical models may be graphically depicted via display device 972 and visually manipulated (for instance, rotated, moved, viewed wholly or partially transparent or semi-transparent, viewed as wireframe or similar images, and/or the like).

In one non-limiting example, the anatomical model generation process may include an image-free process using a point probe (for instance, an optically tracked point probe) to map the actual surface of the target bone(s) that need a new implant. Mapping may be performed after removal of the defective or worn-out implant, as well as after removal of any diseased or otherwise unwanted bone. Points may be collected on the bone surfaces via “painting” by brushing or scraping the entirety of the remaining bone with the tip of the point probe. The collected points may be used to create a three-dimensional model or surface map of the bone surfaces in the computerized planning system. In another non-limiting example, an anatomical model may be mathematically accomplished by capturing a series of Cartesian coordinates that represent the tissue surface, for instance, to generate a model file (for instance, without limitation, *.stl, *.obj., *.fea, *.stp, *.sur, *.igs, *.wrl, *.xyz, and/or the like file formats). In an additional non-limiting example, the anatomical model generation process may include an image-based process based on diagnostic images of the subject anatomy of a patient, such as X-ray images, CT images, and/or the like. Image analysis software may be used to analyze the diagnostic images to generate anatomical models, such as 3D models. In any preceding or subsequent example, the anatomical model generation process may use a combination of image-free and image-based processes. In any preceding or subsequent example, at least a portion of the information used to generate a patella model may include information manually entered by a surgeon (for instance, dimensions). In general, anatomical models according to any preceding or subsequent example may be generated using various processes, including, without limitation, traditional probe painting, 3D imaging mapped with references, visual edge detection, combinations thereof, and/or the like.

For a particular surgical procedure, such as a TKA procedure, the procedure or episode of care can include three phases: pre-operative, intra-operative, and post-operative. During each phase, data may be collected or generated that can be used to analyze the episode of care. Accordingly, display device 972 may operate as an interactive interface that can dynamically update and display how changes to the surgical plan (for instance, visualization of cuts or other patella preparations) would impact the procedure and the final performance of the implant and/or patella.

Computing device (or control system) 910 may include a processor circuitry 920 that may include and/or may access various logics for performing processes according to any preceding or subsequent example, for example, a surgical planning process. For instance, processor circuitry 920 may include and/or may access a computer-assisted surgery logic 922 and/or an anatomical model logic 924. Processing circuitry 920 and/or computer-assisted surgery logic 922, and/or portions thereof may be implemented in hardware, software, or a combination thereof. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution, examples of which may be provided by exemplary computing architecture. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, a control loop, a computational model or application, an AI model or application, an ML model or application, a proportional-integral-derivative (PID) controller, FG circuitry, variations thereof, combinations of any of the foregoing, and/or the like.

Memory unit 940 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 1140 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid-state drive (SSD), and/or the like.

Memory unit 940 may store various types of information and/or applications for performing features and processes according to any preceding or subsequent example, for instance, a surgical planning process according to any preceding or subsequent example. For example, memory unit 940 may store patient information 942, implant information 944, anatomical models 946, surgical plans 950, and/or computer-assisted surgery application 960. In any preceding or subsequent example, all or some of the information depicted as being stored in memory unit 940 may be, in whole or in part, stored in data sources 980a-n and accessible to computing device 910.

In any preceding or subsequent example, patient information 942 may include information for a patient undergoing a surgical procedure being performed via surgical system. Patient information 942 may include personal information (for instance, name, address, and/or the like), physical characteristics (for instance, height, weight, and/or the like), medical information (for instance, health history, health record identifiers, procedure information, and/or the like), and/or any other type of information that may be associated with a patient. In any preceding or subsequent example, patient information 942 may include anatomical information of the patient, such as dimensions, bone hardness, and/or other characteristics of the patient patella.

In any preceding or subsequent example, patient information 942 may include information associated with a population of patients. For example, the population of patients may include individuals that may be used as a comparison with a patient undergoing a procedure in order to generate anatomical models, surgical plans, performance information, and/or the like according to any preceding or subsequent example. In any preceding or subsequent example, computer-assisted surgery application 960 may operate to suggest preparation steps (for instance, cuts, planes, burring, reaming, and/or the like) to achieve certain performance metrics (for instance, kinematics) based on population patent information 942. For example, example, an algorithm, computational model (for instance, a machine learning (“ML”) and/or artificial intelligence (“AI”) model), and/or the like may be trained using population patient information 942 to provide preparation recommendations based on anatomy and outcomes for similar patients.

In any preceding or subsequent example, implant information 944 may include information of an implant associated with a surgical procedure being performed via surgical system 930. In general, implant information 944 may include information required for modeling an implant and/or determining a configuration of an implant on or within patient anatomy. Implant information 944 may include information that may be related to interaction of the patient patella with the implant, for example, to determine patella preparations, performance information, and/or the like.

In any preceding or subsequent example, anatomical models 946 may include virtual models of patient anatomy associated with a surgical procedure being performed via surgical system 930. In any preceding or subsequent example, the virtual models may include mathematical models, graphical models, feature maps, combinations thereof, and/or the like configured to virtually represent patient anatomy within a computing system. In any preceding or subsequent example, anatomical models 946 may be configured to represent a patient patella in accordance with the present disclosure.

In any preceding or subsequent example, a surgical plan 950 may include instructions, method, steps, workflows, and/or the like for performing a surgical procedure. In any preceding or subsequent example, surgical system 930 may develop a surgical plan 950 based on anatomical models 946 and other information specific to the patient (for instance, patient information 942) and/or implant information 944. Surgical plans 950 may provide a recommended patella preparation steps and preparation configurations (for instance, cut information (location, angles, and/or the like), burring information, and/or the like).

Surgical plans 950 may be viewed preoperatively and intraoperatively, and the surgeon can modify surgical plans 950 preoperatively or intraoperatively. Computer-Assisted surgery logic 922, for instance, via computer-assisted surgery application 960, may predict, estimate, or otherwise determine the results of the virtual preparation, such as performance metrics, fit, and/or the like. The surgeon may be presented with the results of the virtual preparation and proceed accordingly (for instance, modifying or removing the virtual preparation, performing a second virtual preparation, and/or the like).

Surgical plans 950 may be administered, in whole or in part, manually by a surgeon, automatically (for instance, computer-or robot-assisted) via computer-controlled surgical instruments 808, and/or combinations thereof. Preceding or subsequent examples are not limited in this context.

In any preceding or subsequent example, computer-assisted surgery logic 922 may operate to perform, implement, or otherwise provide computer-assisted surgery processes for surgical system 930 according to any preceding or subsequent example. For example, in any preceding or subsequent example, computer-assisted surgery logic 922 may operate to manage or control operational features of planning module 912, navigation module 914, control module 916, tracking system 806, and/or surgical instruments 808 described in the present disclosure. In another example, computer-assisted surgery logic 922 may perform, implement, or otherwise provide surgical planning processes according to any preceding or subsequent example.

In any preceding or subsequent example, computer-assisted surgery application 960 may be or may include a software application that includes and/or operates in combination with computer-assisted surgery logic 922 to perform features of surgical system 930 described in the present disclosure. For example, computer-assisted surgery application 960 may operate to present user interfaces via display device 972.

In any preceding or subsequent example, some or all of patient information 942, implant information 944, anatomical models 946, surgical plans 950, computer-assisted surgery logic 922, anatomical model logic 924, and/or a computer-assisted surgery application 960 may be stored in one or more data sources 980a-n accessible to computing device 910 via network 990.

The foregoing description has broad application. While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples. Rather these examples should be considered as illustrative and not restrictive in character. All changes and modifications that come within the spirit of the invention are to be considered within the scope of the disclosure. The present disclosure should be given the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counterclockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular examples. Such terms are not generally limiting to the scope of the claims made herein. Any example or feature of any section, portion, or any other component shown or particularly described in relation to various examples of similar sections, portions, or components herein may be interchangeably applied to any other similar example or feature shown or described herein.

It should be understood that, as described herein, an “example” or “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated examples are to be understood as instances, configurations, specimens, embodiments, illustrations, models, examples, and/or the like (unless otherwise stated), and other manners of embodying the described concepts or features, such as may be understood by one of ordinary skill in the art upon learning the concepts or features from the present disclosure, are within the scope of the disclosure. Furthermore, references to “one embodiment,” “one example,” “preceding or subsequent examples” of the present disclosure are not intended to be interpreted as excluding the existence of additional embodiments or examples that also incorporate the recited features.

In addition, it will be appreciated that while the Figures may show one or more examples of concepts or features together in a single example of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one example can be used separately, or with another example to yield a still further example. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one,” “one or more,” and “and/or,” as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples or configurations. Moreover, the following claims are hereby incorporated into this Detailed Description by this reference, with each claim standing on its own as a separate example of the present disclosure.

What is claimed is:

1. A disposable patella clamp, comprising:
a handle assembly comprising a first handle affixed to a second handle by a hinging mechanism;
an engagement plate disposed on a distal end of the second handle,
a guide assembly disposed on a distal end of the first handle; and
a guidepost extending from a surface of one of the first and second handles, the guidepost configured to interact with an opening formed in the other one of the first and second handles;
wherein when a force is applied to the handle assembly, a patient's patella is secured between the guide assembly and the engagement plate, and the guidepost passes through the opening formed in the handle.

2. The patella clamp of claim 1, wherein the patella clamp is an integral additive manufactured patella clamp.

3. The patella clamp of claim 2, wherein the patella clamp comprises one or more of nylon, biocompatible photopolymer resin (BioMed Clear), thermoplastics, and engineered polymers.

4. The patella clamp of claim 1, wherein the patella clamp is a patient specific patella clamp.

5. The patella clamp of claim 1, wherein the hinging mechanism is a living hinge.

6. The patella clamp of claim 1, the patella clamp further comprising a second guidepost extending from a surface on a proximal end of one of the first and second handles, the second guidepost configured to interact with a second opening formed in the other one of the first and second handles.

7. The patella clamp of claim 6, wherein the hinging mechanism comprises one or more springs.

8. The patella clamp of claim 7, wherein the one or more springs comprise one or more of a coil spring, helical spring, barrel spring, hourglass spring, conical spring, magazine spring, volute spring, flat spring, and torsion spring.

9. The patella clamp of claim 6, wherein the second guidepost comprises a threaded knob, a plate, or a cam configured to interact with the second opening formed in the handle to secure a position of the patella clamp.

10. The patella clamp of claim 1, wherein the guide assembly is a patient specific guide assembly corresponding to patient imaging.

11. The patella clamp of claim 10, the guide assembly comprising a drill guide comprising one or more drill guide openings wherein one or more of a diameter of the drill guide, a layout of the one or more drill guide openings, and a diameter of the one or more drill guide openings are based on the patient imaging.

12. The patella clamp of claim 11, the guide assembly comprising a cutting guide, wherein one or more of a height and a depth of the cutting guide are based on the patient imaging.

13. The patella clamp of claim 1, wherein a central axis of the guide assembly aligns with a longitudinal axis of the handle assembly.

14. The patella clamp of claim 1, wherein a central axis of the guide assembly is disposed at an angle relative to a longitudinal axis of the handle assembly.

15. The patella clamp of claim 14, wherein the central axis of the guide assembly is disposed at a 90-degree angle clockwise or counterclockwise relative to the longitudinal axis of the handle assembly.

16. The patella clamp of claim 1, the engagement plate comprising one or more of teeth, grooves, indentations, and ridges on a surface interior to the patella clamp configured to restrict movement of the patient's patella when secured between the guide assembly and the engagement plate.

17. The patella clamp of claim 1, wherein the guidepost comprises a threaded knob, plate, or cam configured to interact with the opening formed in the other one of the first and second handles to secure a position of the patella clamp.

18. The patella clamp of claim 1, wherein the guide assembly is configured to be interchangeable and affixable to the handle assembly with one or more fastening mechanisms.

19. The patella clamp of claim 1, wherein the engagement plate is configured to be interchangeable and affixable to the handle assembly with one or more fastening mechanisms.

20. A computer-assisted surgical system, comprising:
at least one computing device, comprising:
a display device;
processing circuitry; and
a memory coupled to the processing circuitry, the memory comprising instructions that, when executed by the processing circuitry, cause the processing circuitry to:
receive tracking information associated with a patella detected by at least one tracking element coupled to a tracking patella clamp engaging the patella,
determine patella information based on the tracking information, the patella information indicating at least one characteristic of the patella,
generate a patella model configured as a virtual representation of the patella configured based on the patella information, and
present a graphical user interface (GUI) object visually depicting the patella model on a GUI screen via the display device.
